(19) United States
(12) Patent Application Publication
(10) Pub. No.: US 2025/0027925 A1
(43) Pub. Date: Jan. 23, 2025

(54) METHOD FOR THE DISCOVERY AND DESIGN OF NEW MATERIALS WITHOUT THE GRAVITATIONAL FORCE

(71) Applicant: G-Space, Inc., Sunnyvale, CA (US)

(72) Inventors: Ioana Cozmuta, Sunnyvale, CA (US); Remus Osan, Foster City, CA (US)

(21) Appl. No.: 18/707,086

(22) PCT Filed: Nov. 4, 2022
(86) PCT No.: PCT/US2022/079306

§ 371 Date: May 2, 2024

Related U.S. Application Data
(60) Provisional application No. 63/263,662, filed on Nov. 5, 2021.

(51) Int. Cl. G01N 33/00

(52) U.S. Cl. CPC G01N 33/0078

(57) ABSTRACT

An advanced materials development system predicts stable material formulations and manufacturing processes in reduced-gravity or hyper-gravity environments. A non-contact suspension system may collect experimental data for multiple material samples in a modified-gravity environment. The non-contact suspension system may be equipped with a plurality of laser sources for heating a material sample and stabilizing elements for positioning and stabilizing the material sample in the modified-gravity environment. Data collected from the non-contact suspension system regarding a plurality of material samples may be analyzed and consolidated in a database system of the advanced materials development system. The impact of gravitational force may be determined for a material formulation so that stable material formulations and optimized manufacturing processes may be predicted by the advanced materials development system.

### STATEMENT REGARDING FEDERALLY SPONSORED RESEARCH OR DEVELOPMENT

Some embodiments of this invention were made with United States Government Support under Contract No. 80NSSC20C0375 awarded by the National Aeronautics and Space Administration (NASA) and No. 2015155 awarded by the National Science Foundation (NSF). The U.S. Government may have certain rights in this invention.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in its entirety and for all purposes.

### TECHNICAL FIELD

This disclosure relates to the design of microgravity materials and development, and related manufacturing methods, and more particularly to the design of microgravity materials and manufacturing in a terrestrial environment using non-contact suspension methods.

### BACKGROUND

Materials are often the basis of technological advances in society. For centuries, technologies have relied on terrestrial-based manufacturing of materials and terrestrial-based assembly of structures. However, material properties and manufacturing processes may be limited by the presence of gravity in a terrestrial-based manufacturing process. In-space manufacturing (ISM) has emerged to exploit the differences between properties of materials in space and properties of materials on Earth. A microgravity environment in space can enable higher-quality crystalline structures or new material formulations that are only stable when gravity is not present that are otherwise not stable on Earth. A multitude of materials can benefit dramatically if manufactured in space, such as fiber optic cables, because fiber optic cables can be manufactured with fewer imperfections in space. Manufacture of semiconductors, cathode materials, electrolytes, polymers, medical devices, consumer products, catalytic materials, optical materials, carbon nanotubes and other carbon-derived materials, zeolites, and other materials may be dramatically improved by manufacture in space. However, there is significant economic cost in research, development, and manufacture of products in space.

### SUMMARY

The systems, methods and devices of this disclosure each have several innovative aspects, no single one of which is solely responsible for the desirable attributes disclosed herein.

Provided herein is a method of identifying a predicted material formulation that is stable in a reduced-gravity environment. The method includes receiving, at a computing device, experimental data regarding a plurality of material samples from a non-contact suspension system or from an external data source, processing the experimental data, using the computing device, to determine one or more thermal, mechanical, optical, or structural properties associated with each of the plurality of material samples in a modified-gravity environment, determining an impact of gravitational force on one or more of a material formulation stability, structure, properties, performance, or manufacturing processing, and identifying a predicted material formulation that is stable in a modified-gravity environment.

In some implementations, the one or more thermal, mechanical, optical, or structural properties associated with each of the plurality of material samples in the reduced-gravity environment comprise time-temperature-transition (TTT) diagrams, phase diagrams, and temperature dependent viscosity diagrams. In some implementations, the one or more thermal, mechanical, optical, or structural properties associated with each of the plurality of material samples comprise viscosity, density, surface tension, heat capacity, emissivity, mechanical strength, and thermomechanical stress. In some implementations, the experimental data regarding the plurality of material samples comprise videos and/or images received from a camera on the non-contact suspension system. In some implementations, processing the experimental data comprises segmenting image data into components, mapping segmented components from frame to frame, and extracting feature measurements from the image data to characterize changes in structure and properties of a material sample over time. In some implementations, the method further includes comparing values associated with one or more thermal, mechanical, optical, or structural properties of the material formulation in a reduced-gravity, hyper-gravity, or zero-gravity environment against values associated with one or more thermal, mechanical, optical, or structural properties of the material formulation in a reference-gravity environment. In some implementations, the predicted material formulation achieves one or more desired material properties. In some implementations, the plurality of material samples include a plurality of heavy metal fluoride glasses (HMFGs) each of different compositions. In some implementations, the non-contact suspension system includes primary electrodes in a chamber, where an electric field is generated between the primary electrodes to levitate a material sample, secondary electrodes for stabilizing the material sample in the electric field, a charge source to charge the material sample so that the charged material sample is levitated in the electric field, a plurality of lasers for heating the material sample, and a camera for capturing image data of the material sample. In some implementations, the primary electrodes include an upper electrode and lower electrode in which the material sample is levitated between the upper and lower electrodes, where the secondary electrodes comprise two or more electrodes located in a horizontal plane between the upper and lower electrodes, wherein the charge source comprises a deuterium lamp or ultraviolet (UV) lamp, where the plurality of lasers are configured to heat different regions of the material sample, and where the material sample has a diameter greater than about 3 mm.

Also provided herein is a method of identifying a predicted material formulation that is stable in a reduced-gravity environment. The method includes receiving, at a computing device, experimental data regarding a plurality of material samples from a non-contact suspension system or from an external data source, processing the experimental data, using the computing device, to determine one or more thermal, mechanical, optical, or structural properties associated with each of the plurality of material samples in a modified-gravity environment, determining an impact of gravitational force on one or more of a material formulation stability, structure, properties, performance, or manufacturing processing, and optimizing a manufacturing process or defining a new process for fabricating a material formulation with desired properties in a modified-gravity environment.

In some implementations, the one or more thermal, mechanical, optical, or structural properties associated with each of the plurality of material samples comprise viscosity, density, surface tension, heat capacity, emissivity, mechanical strength, and thermomechanical stress. In some implementations, the experimental data regarding the plurality of material samples comprise videos and/or images received from a camera on the non-contact suspension system. In some implementations, the method further includes comparing values associated with one or more thermal, mechanical, optical, or structural properties of the material formulation in a reduced-gravity, hyper-gravity, or zero-gravity environment against values associated with one or more thermal, mechanical, optical, or structural properties of the material formulation in a reference-gravity environment.

Also provided herein is a non-contact suspension system. The system includes a lower electrode in a chamber, an upper electrode opposite the lower electrode in the chamber. wherein an electric field is configured to be generated between the upper and lower electrodes, two or more auxiliary electrodes located in a horizontal plane between the upper and lower electrodes, wherein the two or more auxiliary electrodes are configured to control a position of a material sample in the electric field, a charge source configured to charge the material sample so that the material sample is levitated in the electric field between the upper and lower electrodes, a plurality of lasers for heating the material sample, and a camera for capturing image data of the material sample.

In some implementations, the non-contact suspension system further includes a first housing enclosing a plurality of material samples each of different compositions, and a second housing holding the plurality of lasers, wherein the plurality of lasers include lasers configured to emit different wavelengths. In some implementations, the material sample has a diameter greater than about 3 mm. In some implementations, the plurality of lasers are configured to heat the material sample at different regions. In some implementations, the non-contact suspension system further includes a computing device coupled to the camera, wherein the computing device includes instructions configured to perform the following operations: receive videos and/or images of the material sample, process the videos and/or images to determine one or more thermal, mechanical, optical, or structural properties associated with the material sample in a modified-gravity environment, and determine an impact of gravitational force on one or more of a material formulation stability, structure, properties, performance, or manufacturing processing. In some implementations, the computing device includes instructions further configured to perform the following operation: identify a predicted material formulation that is stable in a modified-gravity environment, optimize a manufacturing process, or define a new process for fabricating a material formulation with desired properties in the modified-gravity environment. In some implementations, the one or more thermal, mechanical, or structural properties comprise time-temperature-transition (TTT) diagrams, phase diagrams, and temperature dependent viscosity diagrams.

### BRIEF DESCRIPTION OF THE DRAWINGS

FIG. 1 shows a schematic diagram of an example of a prior art electrostatic levitation (ESL) system for suspending a material sample.

FIG. 2A shows a perspective view of various components of an example non-contact suspension system for suspending and investigating material samples according to some implementations.

FIG. 2B shows a chamber of the non-contact suspension system of FIG. 2A according to some implementations.

FIG. 2C shows a plurality of stabilizing spherical elements for stabilizing a material sample in the example non-contact suspension system of FIG. 2A according to some implementations.

FIG. 2D shows a sample cassette for delivery of material samples in the example non-contact suspension system of FIG. 2A according to some implementations.

FIG. 2E shows a housing for either a lamp used for charging a material sample or a laser used for heating a material sample in the example non-contact suspension system of FIG. 2A according to some implementations.

FIG. 3 shows a flow diagram of an example method of identifying a material formulation that is stable in a modified gravity environment or for the discovery or optimization of a manufacturing process for fabricating a material formulation in the modified gravity environment according to some implementations.

FIG. 4A shows a series of images of isothermal dendritic growth experiments (IDGE) from an original video dataset.

FIG. 4B shows an original image of a material sample obtained from a video dataset (4B-1), a segmented image from the original image (4B-2), and graphs showing quantitative time-series analysis of the material sample based on the segmented image (4B-3).

FIG. 4C shows a series of images for detecting coarsening in a solid-liquid mixture, including an original image of a material sample indicating particles by white dots (4C-1), a segmented image of the material sample indicating categorizing properties (e.g., size) of the particles based on color, intensity, and shape (4C-2), and an analyzed image after cluster analysis is performed to combine sets of particles based on similar or dissimilar features (4C-3).

FIG. 4D shows a graph representative of the cluster analysis performed in FIG. 4C.

FIG. 4E shows an example of a generic phase transition diagram of a material formulation with adjustable parameters that are dependent on gravity levels.

FIG. 4F shows an example of a time-temperature-transition diagram that captures the change in stability of a material formulation phase diagram between a 1-g environment and a 0-g environment.

FIG. 5 shows an example of a composition matrix diagram covering a suite of possible material formulations in any percentage combination of three components A, B, and C (AₓB_{y}C_{z}) and indicating their stability in terrestrial-gravity and reduced-gravity environments.

FIG. 6 shows a graph illustrating attenuation at varying wavelengths for fiber optic materials designed and manufactured according to methodologies of the present disclosure in terrestrial-gravity and zero-gravity environments.

FIG. 7 shows an image of an example user interface displaying reports on a material formulation and data regarding an impact of changing levels of the gravitational force on the material formulation.

Like reference numbers and designations in the various drawings indicate like elements.

### DETAILED DESCRIPTION

### Introduction

Space manufacturing has become the frontier of materials research and development. The conditions in space present new opportunities as well as challenges in materials processing and properties. Such conditions in space include long-term microgravity, strong radiation, ultrahigh vacuum, and extreme hot and cold environments that can be exploited in developing an entire new class of materials and corresponding manufacturing methods native to space. Unique materials and novel products can be manufactured in space due to space's harsh environment not found on Earth. Space manufacturing of novel advanced engineered materials can power key technological breakthroughs for a variety of industries such as communications, space exploration, medical devices, storage devices, optical devices, and quantum computing.

By way of an example, certain fiber optic materials such as fluorozirconates (ZBLAN) and fluoroindates (InF) may be manufactured with fewer imperfections in space and provide higher data transmission capability. Gravity can cause small crystals to form that reduce signal strength when drawn into long cables, but a microgravity environment in space can eliminate the presence of such crystals or impurities. These kinds of materials with improved attenuation, flexibility, chromatic dispersion, mechanical strength, etc. may be manufactured with potential breakthrough impact in many industries.

Efforts exist to develop and commercialize products in space. However, this requires heavy capitalization costs in assembling research, manufacturing, and production facilities in space. The cost of transporting materials to space is very high, the manufacturing capabilities in space are limited, and the conducting of research on materials in space is cost-prohibitive. Current space manufacturing approaches are based on trial-and-error, extending the timeline for development of new materials. Thus, laboratory research and development of materials comes with a large initial cost, long timeline, and uncertain payoff. Put simply, the development of advanced novel materials in space may take several years and millions of dollars before commercialization. Using the methodology discussed in the present disclosure, it is possible to discover and design new materials and their corresponding “native-to-space” manufacturing processes in a terrestrial environment to circumvent expensive trial-and-error experiments in space.

The present disclosure offers an advancement over conventional modes of in-space manufacturing by utilizing terrestrial methods to obtain an impact of gravitational force on a specified material formulation's stability, properties, structure, performance, and manufacturing processing. A non-contact suspension system (e.g., electrostatic levitation system) is deployed terrestrially and equipped to suspend material samples in a modified-gravity environment (e.g., zero-gravity environment). The material samples may be observed in the modified-gravity environment and data may be collected regarding the material's thermal, mechanical, optical, and structural properties as well as their manufacturability in the new environment. Data collected by the non-contact suspension system may be integrated with data from other sources and stored in a data management system. Comparing data having values in a modified-gravity environment (e.g., reduced-gravity environment) against data having values in a reference-gravity environment (e.g., terrestrial-gravity environment), an impact of gravitational force on a material formulation's stability, properties, structure, and manufacturing processing can be extracted and quantified. This can be accomplished using machine learning algorithms, computer vision, statistical analysis, and computational modeling. Upon extracting the impact of gravitational force, new material formulations can be identified that are stable in a zero-gravity environment and optimized in manufacturing processes. The uniqueness and innovation in the present disclosure involves the identification of an entirely new class of materials that are only stable in environments beyond the bounds of current stability zones defined by the presence of the gravitational force.

### Non-Contact Suspension System

Non-contact suspension methods may be employed terrestrially to suspend an object and counteract the effects of gravity. These methods may include electrostatic, acoustic, and/or magnetic levitation methods. To date, most advanced systems rely on electrostatic levitation since it is generally applicable to a wider spectrum of materials for processing. Contrary to acoustic levitation that is relatively easy to set up, many acoustic levitation systems are unable to perform materials processing. And contrary to magnetic levitation systems, electrostatic levitation systems are not limited to only processing oxide materials. With electrostatic levitation systems, it is typically challenging to achieve stability of a charged particle since there is no unique minima.

For instance, an electrostatic levitation system may be employed terrestrially to suspend a charged object and counteract the effects of gravity. Usually, a high speed feedback system is typically utilized to provide quasi-static levitation. Electrostatic levitation eliminates or otherwise minimizes gravitational effects to allow materials to be studied without contact with any container or instrumentation. A material in a “softened” state can be maintained in contactless levitation, using an electrostatic levitation system, to simulate a material in a microgravity environment.

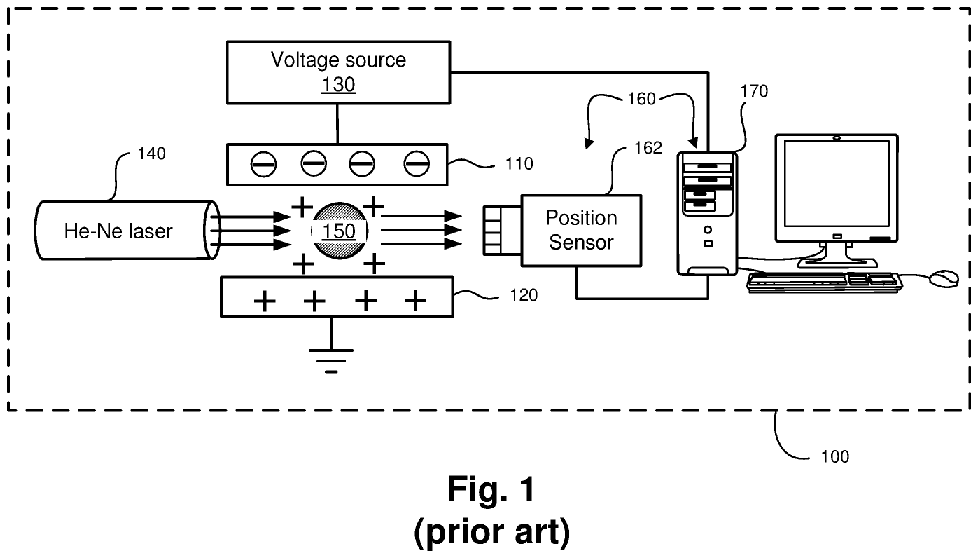

FIG. 1 shows a schematic diagram of an example of a prior art electrostatic levitation system for suspending a material sample. An electrostatic levitation system 100 includes a pair of electrodes 110, 120 located above and below a material sample 150. The pair of electrodes 110, 120 may surround the material sample 150, which may be an electrostatically charged object. A high voltage source 130 is electrically connected to a top electrode 110 and applies a high voltage to the top electrode 110 while a bottom electrode 120 is connected to ground. The material sample 150 and the bottom electrode 120 may be the same polarity to repel against the force of gravity. The Coulomb force between the electrostatically charged object and the surrounding electrodes 110, 120 suspends the material sample 150 against the force of gravity. A feedback system 160 includes a position sensor 162 that senses the position of the material sample 150 and delivers this information to a computing device 170. By way of an example, the position sensor 162 may include a charge coupled device (CCD) camera to capture an image of the material sample 150. When the material sample 150 moves above or below a desired position, the computing device 170 is in communication with the high voltage source 130 to adjust the voltage applied to the top electrode 110. The computing device 170 may be programmed to not only control the voltage applied to the top electrode 110, but may adjust the rate of variation to account for oscillations of the material sample 150. In FIG. 1. the electrostatic levitation system 100 can further include a laser source 140 to heat up the material sample 150. The laser source 140 may emit a collimated laser beam to melt the material sample 150 so that the material sample 150 can be suspended in a liquid phase for contactless processing. Various properties such as thermophysical properties of the material sample 150 can be studied by heating the material sample 150 with the laser source 140. For instance, the laser source 140 may be a helium-neon laser source.

Existing electrostatic levitation systems are limited in scale and scope. Existing electrostatic levitation systems may perform contactless processing for samples of limited size, such as samples having a diameter equal to or less than about 3 mm (e.g., between 1 mm and 3 mm). Furthermore, such systems may not be able to stabilize samples within a desired tolerance. For example, positioning systems in these systems may be unable to precisely stabilize samples against horizontal movements (x-y direction) or rotational movements. In addition, existing electrostatic levitation systems may be constrained to a laser source for heating only certain materials to within a limited temperature range. Some materials may be transparent to a laser wavelength of a particular laser source. Moreover, existing electrostatic levitation systems are generally unable to process multiple samples efficiently, thereby limiting throughput and scalability. Existing electrostatic levitation systems are also limited in their ability to process data collected by various instrumentation and to predict properties, performance, stability, and manufacturing processes of certain materials when they are in outer space.

A non-contact suspension system of the present disclosure addresses some of the foregoing deficiencies in existing levitation systems. The non-contact suspension system of the present disclosure may be configured to process samples of relatively large sizes, stabilize samples within a tight tolerance, heat a variety of samples according to a suitable laser wavelength, process multiple samples efficiently, and process collected data to accurately predict properties, performance, stability, and manufacturing processes of material formulations in outer space.

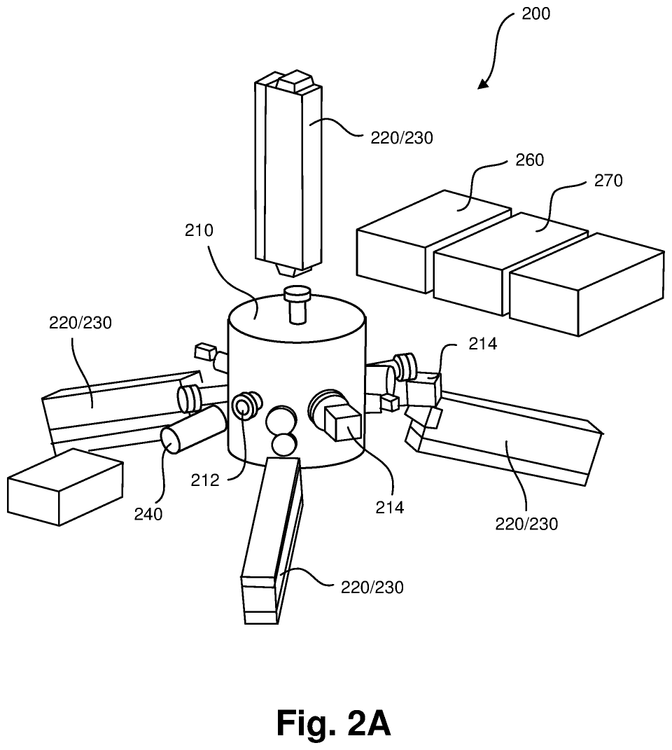

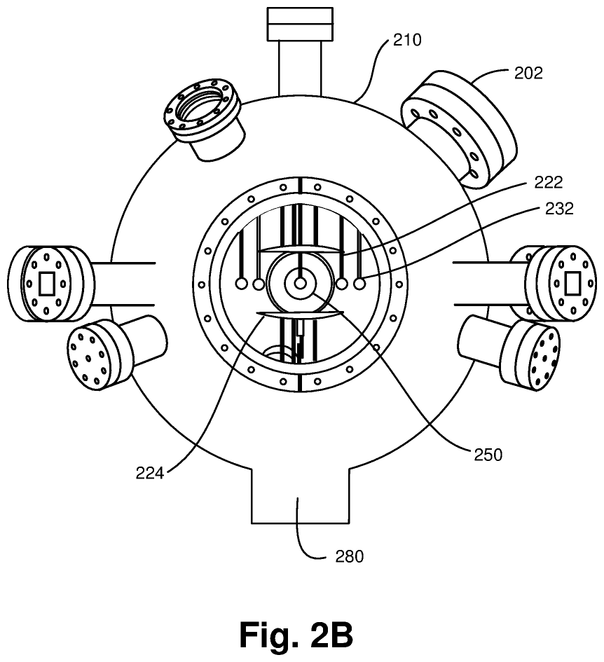

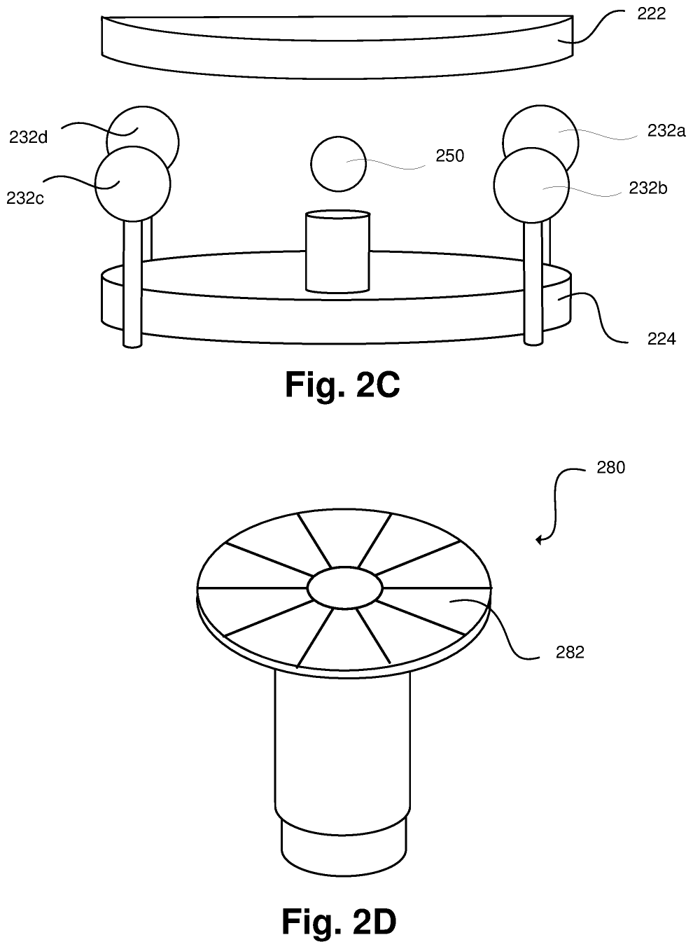

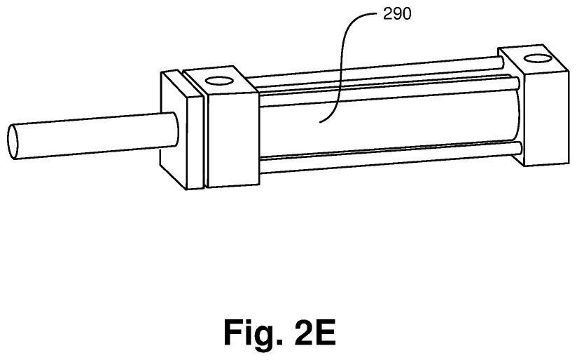

FIGS. 2A-2E illustrate an example non-contact suspension system that can be implemented in the present disclosure. In some implementations, the non-contact suspension system is an electrostatic levitation system. However, it will be understood that other non-contact suspension systems may be employed. In some other implementations, the non-contact suspension system is an optical levitation system, magnetic levitation system, or other multifunctional levitation system.

FIG. 2A shows a perspective view of various components of an example non-contact suspension system for suspending and investigating material samples according to some implementations. While the non-contact suspension system 200 may be illustrated as an electrostatic levitation system, it will be understood that the non-contact suspension system 200 may be implemented as an optical levitation system, magnetic levitation system, or other suitable suspension system. The non-contact suspension system 200 includes a chamber 210 along with a variety of instrumentation that performs functions such as positioning, charging, stability and control, heating, imaging recording, and thermal measurements. The chamber 210 may be a vacuum chamber such as a stainless steel vacuum chamber. The chamber 210 provides an enclosed space that holds one or more material samples. In some embodiments, the chamber 210 may be a cylindrical or spherical chamber, though it will be understood that the chamber 210 may be any suitable three-dimensional geometry. In some implementations, the chamber 210 may have dimensions between about 5 inches and about 30 inches in diameter and/or height, or between about 8 inches and about 15 inches in diameter and/or height. As shown in FIG. 2A, the chamber 210 may have one or more viewports 212 for viewing an interior of the chamber 210 from the external environment. In some embodiments, the chamber 210 may have mechanical feedthroughs for providing coupling to a mechanical or vacuum pump. In some embodiments, the chamber 210 may have electrical feedthroughs for providing coupling to electrical components such as electrodes. The non-contact suspension system 200 may be used in conjunction with a UV or other sources of radiation to replicate characteristics of space environments such as a combination of vacuum, cold, and low temperatures and radiation.

The chamber 210 may be depressurized to low pressures (e.g., 1×10⁻¹¹ atm or lower) using a vacuum pump, or at least configured to operate in inert gas atmosphere. In some embodiments, the chamber 210 may be operated at a chamber temperature that is constant with the ambient external temperature. In some embodiments, the chamber 210 may be operated and maintained at an internal chamber pressure of 1×10⁻¹¹ atm or less. That way, the material sample in the chamber 210 avoids contamination. The chamber 210 may further include ports 214 for coupling various instrumentation to the chamber 210. For instance, some ports 214 may provide coupling for heating sources 220 such as laser sources. Some ports 214 may provide coupling for charging sources 230, which may be applicable in electrostatic levitation systems. Some ports 214 may provide coupling for various sensors 240 including image sensors, position sensors, pressure sensors, temperature sensors or pyrometers, etc. The non-contact suspension system 200 may optionally include a voltage source 260 such as a voltage amplifier for supplying power to electrodes in the chamber 210 and generate an electric field between the electrodes, particularly where electrostatic levitation systems are utilized. A material sample can be levitated between the electrodes inside the chamber 210. Alternatively, the material sample can be levitated between mirrors and/or magnets. The non-contact suspension system 200 may further include a computing device 270 for controlling operations of the non-contact suspension system 200 and capturing data from experiments conducted on material samples in the chamber 210.

FIG. 2B shows a chamber of the example non-contact suspension system of FIG. 2A according to some implementations. Within the chamber 210, an upper stabilizing element 222 and a lower stabilizing element 224 are included. A material sample 250 is provided between the upper stabilizing element 222 and the lower stabilizing element 224. In some implementations, the stabilizing elements 222, 224 are electrodes. In some implementations, the stabilizing elements 222, 224 may be mirrors and/or magnets. Where the stabilizing elements 222, 224 are electrodes, the optional voltage source 260 may be electrically connected to either the upper electrode 222 or the lower electrode 224 so that an electric field may be generated between them. When the material sample 250 is electrostatically charged, the electric field between the upper electrode 222 and the lower electrode 224 may counteract the force of gravity to suspend or levitate the material sample 250 inside the chamber 210. In some embodiments, each of the upper electrode 222 and the lower electrode 224 may be parallel horizontal plates, though the plates may have some curvature to ensure that a viable electric field is generated. In some implementations, each of the upper electrode 222 and the lower electrode 224 may have a diameter between about 10 mm and about 50 mm, or between about 15 mm and about 30 mm. In some implementations, a distance between the upper stabilizing element 222 and the lower stabilizing element 224 is between about 5 mm and about 30 mm, such as about 12 mm. The upper stabilizing element 222 and the lower stabilizing element 224 may serve as the “primary stabilizing elements” in suspending the material sample 250 in the chamber 210 against the force of gravity.

The material sample 250 being investigated in the non-contact suspension system 200 may be any suitable material including electrically conducting, semiconducting, or insulating material. Suitable materials may be electrostatically charged in an electrostatic levitation system, and different types of materials may accumulate and distribute charge more easily than others. For example, metallic samples are easier to charge than insulating samples because charge distributes naturally in metallic samples. The material sample 250 may be extracted from a sample cassette 280 to provide the material sample 250 between the upper stabilizing element 222 and the lower stabilizing element 224. A robotic arm may be utilized to deliver the material sample 250 to a desired location (e.g., equilibrium position) in the chamber 210. The material sample 250 is generally spherical in shape. The material sample 250 may be kept in its solid or liquid state without contacting any walls, container, or instrumentation. In some embodiments, the material sample 250 is between about 1 mm and about 10 mm in diameter, between about 2 mm and about 8 mm in diameter, or between about 3 mm and about 5 mm in diameter. Alternatively, the material sample 250 is between about 1 cm and about 10 cm in diameter, between about 2 cm and about 8 cm in diameter, or between about 2 cm and about 5 cm in diameter. For such larger samples, the dimensions of the chamber 210 and associated components may be larger. For example, each of the upper stabilizing element 222 and the lower stabilizing element 224 may have a diameter between about 10 cm and about 50 cm, or between about 15 cm and about 30 cm.

Within the chamber 210, a plurality of positioning stabilizing elements 232 are included. The positioning stabilizing elements 232 may be electrodes in some implementations. Alternatively, the positioning stabilizing elements 232 may be mirrors and/or magnets in some other implementations. In some embodiments, each of the plurality of positioning stabilizing elements 232 are spherical in shape, though it will be understood that other suitable geometries may be used. Rounded surfaces of the positioning stabilizing elements 232 avoid sharply curved surfaces that could generate arc discharges when high voltages are applied. One or more of the plurality of positioning stabilizing elements 232 may be electrically connected to the optional voltage source 260. The plurality of positioning stabilizing elements 232 may be spaced apart equidistant from each other. In one example, the plurality of positioning stabilizing elements 232 may be arranged in a tetrahedral configuration or other polyhedral configuration about the material sample 250. In another example, the plurality of positioning stabilizing elements 232 may be arranged in a triangular, circular, square, or other geometric arrangement about the material sample 250. The plurality of positioning stabilizing elements 232 may serve as “secondary stabilizing elements” in positioning the material sample 250 in the chamber 210. The positioning stabilizing elements 232 may serve to restrain sample translational and rotational motion in the chamber 210.

FIG. 2C shows a plurality of stabilizing elements (e.g., stabilizing spheres) for stabilizing a material sample in the example non-contact suspension system of FIG. 2A according to some implementations. Stabilizing elements used in stabilizing and positioning a material sample 250 in the chamber 210 can include primary stabilizing elements 222, 224 and secondary stabilizing elements 232a, 232b, 232c, 232d. To levitate the material sample, at least the primary stabilizing elements 222, 224 are used. In some embodiments, the upper stabilizing element 222 may be hemispherical and the lower stabilizing element 224 may be flat. The upper stabilizing element 222 is positioned above the material sample 250 and the lower stabilizing element 224 is positioned below the material sample 250. The secondary stabilizing elements 232a, 232b, 232c, 232d may be positioned in a horizontal plane between the upper stabilizing element 222 and the lower stabilizing element 224. Each of the secondary stabilizing elements 232a, 232b, 232c, 232d are arranged at opposite corners of a square configuration. Each of the secondary stabilizing elements 232a, 232b, 232c, 232d may be equally spaced apart from the material sample 250 or at least from a center position defined by the primary stabilizing elements 222, 224 and secondary stabilizing elements 232a, 232b, 232c, 232d. Each of the primary stabilizing elements 222, 224 and the secondary stabilizing elements 232a, 232b, 232c, 232d may be connected to an electrical ground. The optional voltage source 260 may vary the voltages applied to the primary electrodes 222, 224 and the secondary electrodes 232a, 232b, 232c, 232d to control a position of the material sample 250 in the chamber 210. The polarity of the material sample 250 may have the same or opposite polarity of the voltage applied to any of the electrodes. In some embodiments, the same polarity is applied to each of the primary electrodes 222, 224 and the secondary electrodes 232a, 232b, 232c, 232d and to the material sample 250.

Though the stabilizing elements in FIG. 2C are particularly shaped and particularly arranged for stability and control of the material sample 250, it will be understood that other shapes and other arrangements may be used. By way of an example, a dish electrode setup can include two parallel horizontal plates for stability and control of the material sample 250. In another example, a ring electrode setup can include two central circular disk electrodes surrounded with ring-shaped electrodes on the periphery for position control. In yet another example, a tetrahedral electrode setup can include four spherical electrodes in a tetrahedral configuration. In still yet another example, a primary and secondary electrode setup can include two parallel dish-shaped electrodes with through-holes as well as four secondary electrodes. The through-holes may facilitate sample delivery.

The position of the material sample 250 may correspond to a position in an X, Y, Z coordinate system. The position of the material sample 250 may be sensed by two or more position sensors. The two or more position sensors may be part of an imaging system for monitoring the location and position of the material sample 250 in real-time. A position controller or computing device detects and receives a position of the material sample 250 and uses an active feedback control algorithm to adjust the position of the material sample 250 by varying voltages applied to at least each of the secondary stabilizing elements 232a, 232b, 232c, 232d. The position controller may provide time resolution on the order of a few milliseconds or less (e.g., 0.5 to 1 ms time resolution). Since there is no stable minimum in terms of position between the primary stabilizing elements 222, 224, the secondary stabilizing elements 232a, 232b, 232c, 232d are used to avoid translational and rotational motion of the material sample 250. That way, a computing device can actively and precisely suspend the material sample 250 in a desired location in the X, Y, Z coordinate system by applying an active feedback control algorithm using the position sensors and the primary stabilizing elements 222, 224 and the secondary stabilizing elements 232a, 232b, 232c. 232d.

FIG. 2D shows a sample cassette for delivery of material samples in the example non-contact suspension system of FIG. 2A according to some implementations. The sample cassette 280 may include a housing or container that stores a plurality of material samples 250. The sample cassette 280 may include a plurality of storage units 282. The sample cassette 280 may include at least 5, at least 10, at least 12, at least 15, or at least 20 storage units 282 so that multiple material samples 250 may be processed and studied in succession. This improves efficiency and throughput so that material samples 250 do not have to be loaded and unloaded individually from the chamber 210 each time. In some implementations, the sample cassette 280 includes a spin table where some samples are locked in place during processing. A belt drive can be used to rotate the samples and hydraulic cylindrical push rods can house the samples and position them on the lower stabilizing element 224.

In some embodiments, the sample cassette 280 may be positioned below the lower stabilizing element 224 of the chamber 210. The material sample 250 may be delivered to an equilibrium position of the chamber 210 via a robotic arm. Linear motion of the robotic arm may be induced by actuators. In some embodiments, the material sample 250 rolls down a shoot ramp to get to an initial position on the lower stabilizing element 224 prior to being placed in the equilibrium position. Especially for insulating materials, an initial charge may be accumulated and distributed on the material sample 250 as a result of friction on the shoot ramp. The initial charge may be sufficient for the material sample 250 to be levitated in an electrostatic levitation system. The material sample 250 is subsequently charged for levitation in the electric field by thermionic emission or photoelectric charging by a UV source.

FIG. 2E shows a housing for instrumentation used for charging a material sample or heating a material sample in the example non-contact suspension system of FIG. 2A according to some implementations. An instrument 290 used for charging the material sample 250 may be a charge source 230 such as an intense UV source. The charge source 230 may provide sufficient intensity to maintain a sufficient charge on the material sample 250 so that the material sample 250 can interact with the surrounding electric field. The charge source 230 may ensure that the charge on the material sample 250 is relatively constant even in prolonged heating. In some embodiments, the charge source 230 is a deuterium arc lamp. An instrument 290 used for heating the material sample 250 may be a laser source 220. For example, a laser source 220 can be a Nd:YAG laser source, Er:YAG laser source, fiber laser source, CO₂ laser source, or other laser light sources. CO₂ lasers may be useful for heating insulating materials, and Er:YAG lasers may be useful for heating conductive materials. Direct diode lasers may be useful for superficial heating. Multiple laser sources 220 may be mounted to the chamber 210 at different positions to heat the material sample 250 from different angles and to heat the material sample 250 at different regions. By having a larger sample size, e.g., greater than about 3 mm in diameter, the multiple laser sources 220 may be configured in positions to heat different zones, regions, or hemispheres of the material sample 250. By being able to heat different zones, regions, or hemispheres differently, users can extract thermomechanical/thermophysical data from the material sample 250. In some implementations, a plurality of laser sources 220 may be housed in a turret or rotating head. This allows laser sources 220 of different wavelengths to be easily changed depending on a material of the material sample 250 being processed. Such laser sources 220 may include fiber lasers or other laser light sources. Instead of mounting and dismounting a different laser when a different material is being processed, the turret or rotating head allows a user to switch to an appropriate laser source 220 with an appropriate wavelength based on the material of the material sample 250 being processed. This further increases efficiency and throughput for processing material samples 250. The laser sources 220 are configured to heat the material sample 250 to a desired temperature, or at least certain regions of the material sample 250 to a desired temperature, so that thermomechanical/thermophysical data regarding the material sample 250 suspended in the non-contact suspension system 200 can be extracted. One or more temperature sensors such as pyrometers may be used to monitor the temperature of the material sample 250 during heating. In some cases, the material sample 250 is heated to a liquid state. Another instrument 290 used in the non-contact suspension system 200 may include one or more imaging lasers, where the one or more imaging lasers may project images onto the one or more position detectors. In some implementations, the one or more imaging lasers can be a He—Ne laser source. CCD cameras may be used to capture the position and image of the material sample 250 in the chamber 210.

Sample measurements may be performed using a combination or suite of instruments 290 in the non-contact suspension system 200. The combination of instruments 290 in the non-contact suspension system 200 may provide positioning, charging, stability and control, heating, imaging, and thermal measurements of the material sample 250. By way of an example, the combination of instruments 290 may include temperature sensors and imaging sensors (e.g., high-resolution cameras) to obtain mechanical, optical, structural, and thermal measurements. The material sample 250 may be heated to desired temperatures using one or more laser sources 220, or the material sample 250 may simply be melted using the one or more laser sources 220. Sample measurements and sample imaging may occur as the material sample 250 is heated. Thermal measurements may be collected to obtain a viscosity, density, surface tension, emissivity, heat capacity, or other sample property measurement of the material sample 250 when the material sample 250 is heated. In some cases, an AC voltage pulse may be sent at a certain frequency to generate oscillations on the surface of the material sample 250 for viscosity measurements. For viscosity measurements, the material sample 250 has to reach a certain temperature such that it results in material softening. For density measurements, edge detection or pixel counting is used to determine an area of the material sample 250 in lieu of volume. Videos and/or images may be taken of the material sample 250 when performing sample measurements to be stored as a database record in a database system or other data repository. The videos and/or images may be segmented, mapped, and analyzed frame-by-frame to extract relevant feature information. Such operations may be executed by computer vision. Other measurements or data collected from the material sample 250 when suspended in the non-contact suspension system 200 may include but are not limited to mechanical fragility, reflectivity, melting, time-temperature-transition (TTT) diagrams, phase diagrams, and temperature-dependent viscosity diagrams. The collected data may be organized in various fields or categories, and may exist in one or more database records in one or more database systems. The data may be stored and organized in the one or more database systems, where the data may be integrated with reference data taken from external data sources. In some implementations, algorithmic operations may be applied using machine learning to determine properties, performance, and stability of certain material formulations in microgravity environments.

After sample measurements are taken, laser sources 220 are turned off and the material sample 250 is cooled. The charge source 230 may also be turned off and the voltage source 260 may also be turned off so that the material sample 250 may be recuperated through the lower stabilizing element 224. Additional samples may be investigated by the non-contact suspension system 200 by rotating through material samples 250 in the sample cassette 280.

### Predicting Material Properties, Performance, and Stability in Modified-Gravity Environments for New Material Discovery

Technological advancements are often driven by development of new materials or existing materials with improved properties. Many advancements in materials research and development are taking place in space, but such research and development may be time-consuming and cost-prohibitive. The present disclosure enables advancements in materials research and development terrestrially by investigating materials in microgravity environments and using computational tools to understand the impact of gravity on material properties, performance, structure, and stability. As current space manufacturing approaches are based on trial-and-error and take longer to scale up, the present disclosure can terrestrially identify new stable materials in microgravity environments and predict optimal space manufacturing envelopes. This cuts development time, reduces testing iterations, produces innovative materials faster, and provides significant savings in cost.

Material properties, performance, structure, and stability may be different depending on the gravitational impact of the environment. The gravitational force in an environment may be measured in terms of gravitational force equivalent (g-force), where 1-g corresponds to a gravitational acceleration of 9.81 m/s² on Earth at sea level. The g-force on the moon is about 0.166-g and the g-force in free space in outer space is approximately 0-g. Material properties in a 1-g environment may be different than material properties in a 0-g. partial-g, or hyper-g environment, and manufacturing materials in a 1-g environment may result in different properties compared to manufacturing the same materials in a 0-g, partial-g, or hyper-g environment. Thus, gravity is a tunable parameter in material design.

The impact of gravitational force on material properties, performance, structure, stability, and manufacturing processes can be determined as a quantifiable parameter. As used herein, this parameter may be referred to as “gravitational impact.” The gravitational impact may be determined by a set of computer vision, data science, computational algorithms, and physical models that extract the impact of gravitational force on material properties, performance, structure, stability, and manufacturing processes based on a comparison between terrestrial reference (e.g., 1-g) data values and non-terrestrial (e.g., 0-g) data values. In fact, determining the gravitational impact can be made by comparing data values obtained from at least two different gravitational environments. As used herein, a “reduced-gravity environment” refers to an environment where the gravitational force is less than 1-g, a “hyper-gravity environment” refers to an environment where the gravitational force is greater than 1-g, and a “modified-gravity environment” refers to either a hyper-gravity environment or reduced-gravity environment.

In the present disclosure, an “advanced materials development system” refers to software and/or computational logic for performing one or more functions associated with materials development in different gravitational environments. Such functions include identifying material formulations that are stable in zero-gravity, reduced-gravity, or hyper-gravity environments, calculating the impact of gravity on material formulation stability, structure, properties, performance, and manufacturing processing, and predicting optimal manufacturing envelopes for material formulations in different gravitational environments. An advanced materials development system may contain or may be configured to interact with a database systems containing materials data for a plurality of material formulations. In some implementations, the computational logic for identifying material formulations that are stable in modified-gravity environments, calculating the impact of gravity on material formulation stability, structure, properties, performance, and manufacturing processing, and predicting optimal manufacturing envelopes for material formulations in different gravitational environments, may reside and/or may execute on one or more remote servers, on a user device, or on a computing device that may serve as an intermediary between a remote server and the user device.

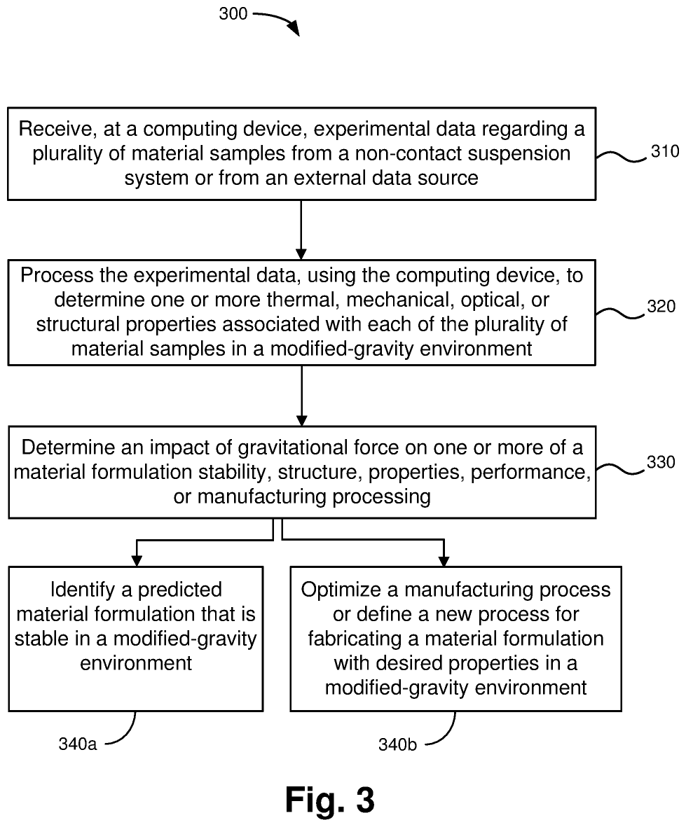

FIG. 3 shows a flow diagram of an example method of identifying a material formulation that is stable in a modified-gravity environment or optimizing a manufacturing process associated with a material formulation in the modified-gravity environment according to some implementations. The operations in a process 300 may be performed in different orders and/or with different, fewer, or additional operations. At least some of the operations of the process 300 are performed using the advanced materials development system.

At block 310 of the process 300, experimental data regarding a plurality of material samples from a non-contact suspension system or from an external data source is received at a computing device. The computing device may be a server, a user device, or an intermediary between a remote server and a user device. The computing device may be in electronic communication with a database or database system containing materials data for a plurality of material formulations. The database system may include one or more servers, which may be dedicated or leased such as via cloud computing resources. The experimental data regarding the plurality of material samples may be stored and organized in the database system.

Data is stored in the database system and may be accessible by one or more users. Data may be stored in different objects such as database records. The database system can handle creation, storage, organization, and/or access to the database records. Materials data regarding various material formulations may be stored as database records or other accessible record. Materials data for various material formulations may include but are not limited to datasets of experimental results, datasets from experimental simulations, datasets from physical models, and datasets of materials data sheets or properties retrieved from external data sources. In some cases, the materials data for various material formulations may further include images and/or videos associated with material formulations, where such images and/or videos may be analyzed to extract relevant features regarding a material formulation. Accordingly, some of the materials data for the various material formulations may be presented in an unstructured or raw format, and some of the materials data for the various material formulations may be presented in a structured or tabulated format. At least some of the materials data may include metadata or data organized according to various fields and categories. Metadata may contain material formulation, properties associated with the material formulation, and experimental conditions such as the gravitational force of the environment from which the properties are extracted.

The database system provides a comprehensive materials library of materials data in various gravitational environments. This enables users to gain access to experimental data, simulated data, and known data regarding a collection of material formulations obtained in a microgravity environment, terrestrial gravity environment, or other gravity environment. The database system may include data obtained from external data sources. By way of an example. an external data source may include the National Aeronautics Space Administration (NASA) Physical Sciences and Informatics and/or GeneLab databases, which may include data obtained from microgravity experiments. By way of another example, an external data source may include the BASF and 3M product databases that contains a comprehensive list of material products from a series of industries. Data from the aforementioned databases may be pulled, integrated, and consolidated with the database system of the advanced materials development system. The database system of the advanced materials development system may integrate its own database records with one or more external data sources.

The advanced materials development system may collect and/or process the experimental data received at the computing device. The advanced materials development system may store and organize the experimental data in the database system in electronic communication with the computing device. Any experimental data from the non-contact suspension system may be provided to the database system in addition to or in the alternative to experimental data from third-party repositories or external data sources. Accordingly, experimental data may be integrated with materials data from various data sources in the database system. Materials data from external data sources may include thermal, mechanical, optical, and/or structural properties associated with one or more materials in reference-gravity (e.g., terrestrial-gravity) environments. Such materials data in reference-gravity environments may be compared against experimental data collected in modified-gravity environments.

The experimental data regarding a plurality of material samples may be received at the computing device in a raw or unstructured format. In some embodiments, the experimental data is in the form of images and/or videos. Though the experimental data may be stored and organized in the database system of the advanced materials development system, some or all of the experimental data may need to be processed into a suitable data ingestion format. In some cases, some or all of the experimental data may be processed by the advanced materials development system into a format that is applicable in machine learning algorithms.

Some of the datasets of the experimental data may be received from the non-contact suspension system. Some of the datasets of the experimental data may be received as images and/or videos over various wavelength spectra, including but not limited to infrared, visible. and ultraviolet spectra. Some of the datasets of the experimental data may be received as measurements. Some of the datasets of the experimental data may be received as existing datasets from microgravity platforms (parabolic, suborbital, orbital, etc.).

At block 320 of the process 300, the experimental data is processed using the computing device to determine one or more thermal, mechanical, optical, or structural properties associated with each of the plurality of material samples in a modified-gravity environment. In some embodiments, the modified-gravity environment is a reduced-gravity environment or zero-gravity environment. In some embodiments, the modified-gravity environment is a hyper-gravity environment. The one or more thermal, mechanical, optical, or structural properties may be extracted from the experimental data using the advanced materials development system. In some implementations, the plurality of material samples may include heavy metal fluoride glasses of different compositions.

If the experimental data is in the form of images or videos, then the images or videos may be processed to extract relevant information. For instance, different measurements may be extracted from images and/or videos of the material samples, including but not limited to viscosity, density, emissivity, and surface tension. This enables the database system to receive processed experimental data from the non-contact suspension system so that thermophysical properties or other material properties in a microgravity environment may be provided. Such properties may be analyzed across multiple material formulations under different gravitational environments using the advanced materials development system.

A non-contact suspension system described in FIG. 2A-2E may levitate material samples. While levitated, the material samples in a microgravity environment are studied and evaluated according to certain processing conditions. Thermophysical properties, optical properties, and other material properties in the microgravity environment can be extracted. In some implementations, the levitated material samples may undergo temperature changes and possible phase transitions. In some instances, the levitated material samples may be melted, cooled, supercooled, and solidified. In some implementations, images and/or videos of the levitated material samples may be recorded at different temperatures over time. Image processing may involve detection, segmentation, and feature extraction. Image processing may involve detection, segmentation, and feature extraction using computer vision-based data science techniques. Features that may be extracted from image processing may include but are not limited to size, center of mass, and degree of asymmetry. Video processing may involve image processing on frames, mapping of segmented components from frame to frame, and time series extraction of feature measurements, among other things. Video processing may involve image processing on frames, mapping of segmented components from frame to frame, and time series extraction of feature measurements using computer vision-based data science techniques. In one example, video processing may be used for isothermal dendritic growth experiments (IDGE) to study and measure growth velocity and tip radii of solidifying materials in a microgravity environment through a supercooling process. In some implementations, a force may be applied to one or more material samples in a liquid state to induce oscillations. The rate of decay of the amplitude of the oscillation and the frequency of the oscillation are determined to ascertain the temperature dependence of viscosity with respect to surface tension. Video processing showing changes in center of mass and changes in the degree of asymmetry may assist in calculating viscosity. In some implementations, such image processing and video processing may be performed by the advanced materials development system. Extracted feature information from image and video processing may be stored in the database system. The one or more thermal, mechanical, optical, or structural properties may be

extracted from the videos and/or images of the experimental data. Such experimental data collected from the non-contact suspension system, including time-dependent measurements extracted from videos and/or images, may be transformed into material properties such as viscosity, density, emissivity, surface tension, and time-temperature-transformation (TTT) diagrams by the advanced materials development system. Time-series analysis may be performed by the advanced materials development system to quantify dynamic processes to characterize changes in structure and properties over time. The experimental data may be collected and transformed into a standardized format to ensure consistency across datasets.

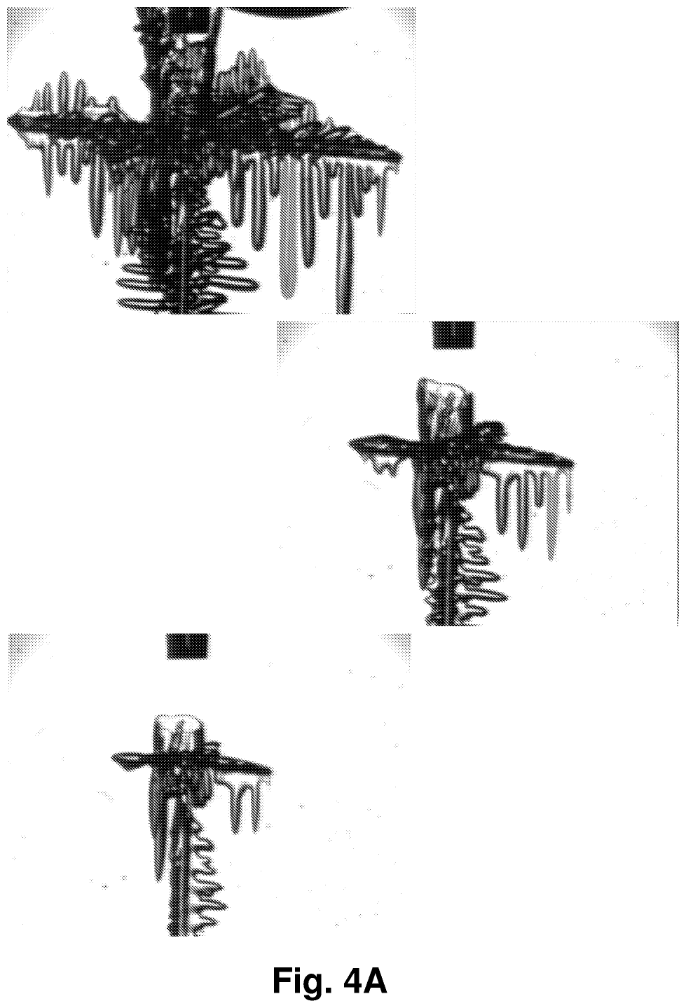

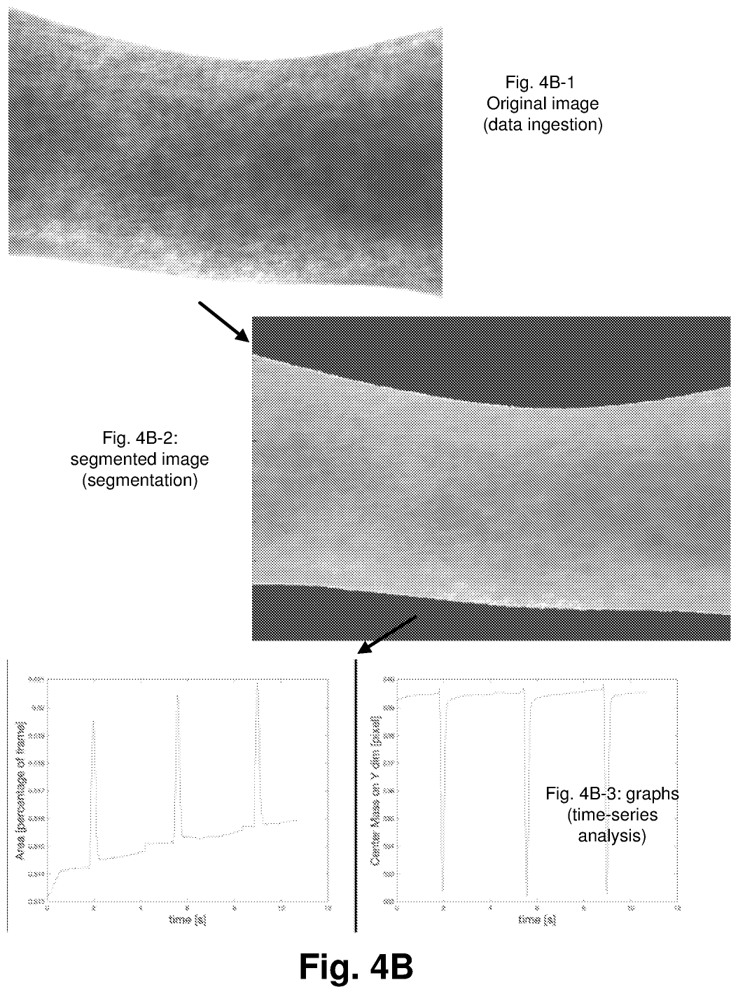

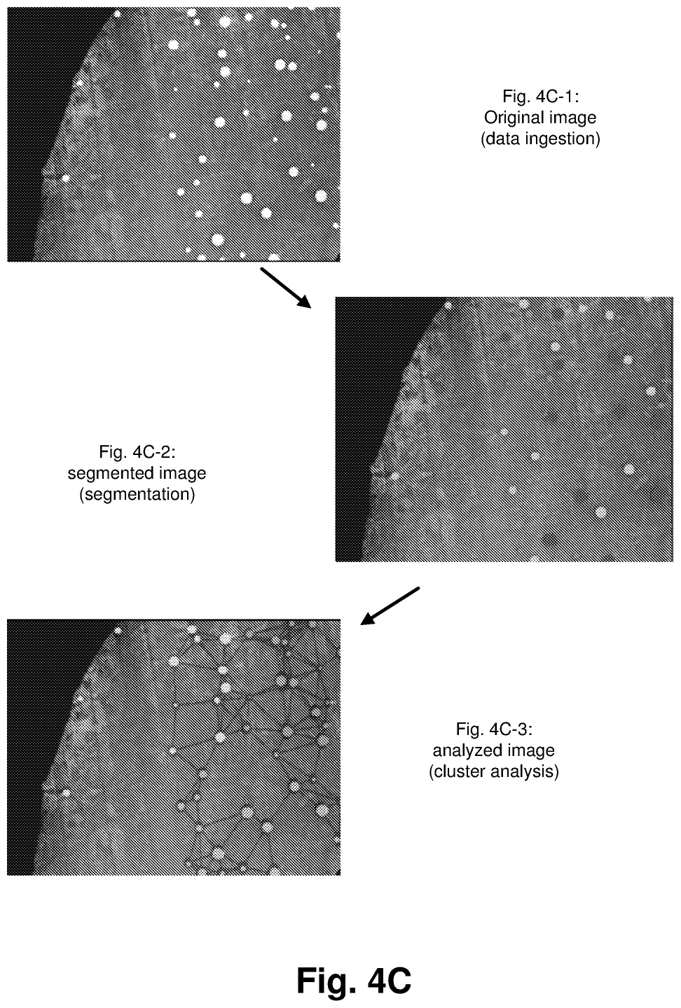

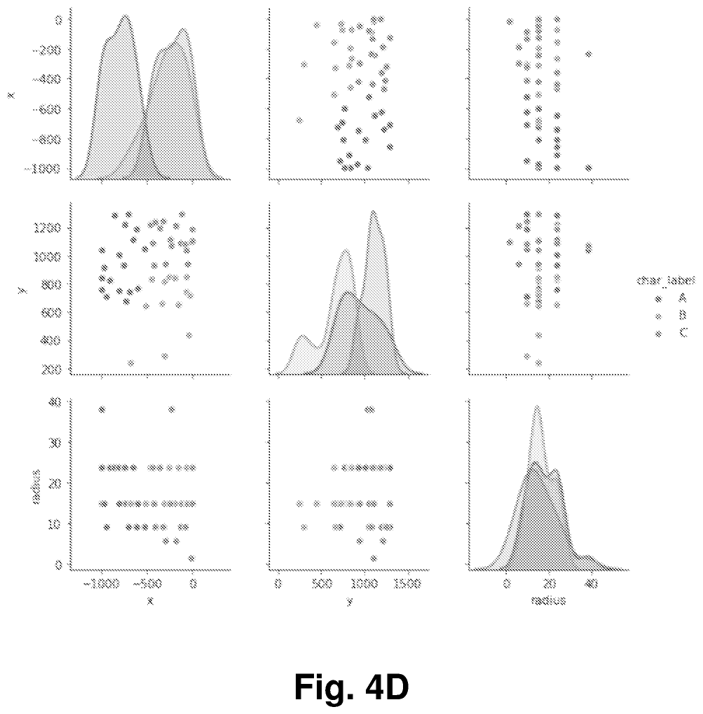

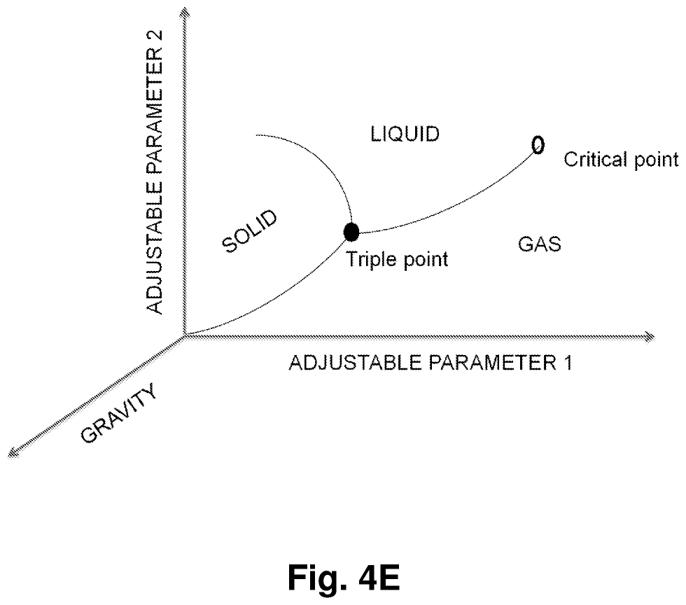

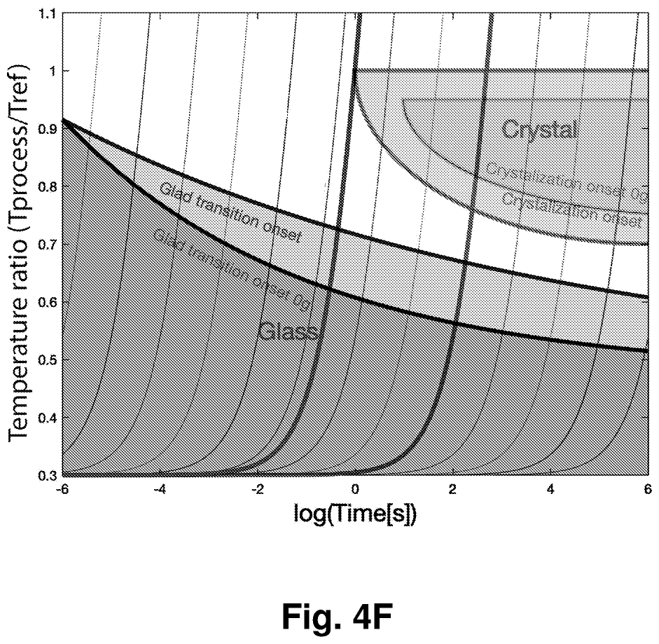

FIG. 4A shows a series of images of isothermal dendritic growth experiments (IDGE) from an original video dataset. Images from IDGE video datasets may be used to study and measure growth velocity and tip radii of solidifying materials in microgravity through a supercooling process. Images from IDGE video datasets shown in FIG. 4A are publicly available data. Crystal dendrites form diverse structural patterns with solidifying molten materials which can affect various properties such as ductility, strength, electrical conductivity, and welding ability. Such properties are directly related to the ability to predict size and shape of dendrite growth. Some of the quantitative properties of interest are dendritic growth velocity and tip radii. Dendritic growth and properties affected by crystal dendrites may be dependent on gravity. A time series analysis performed by computer vision in the advanced materials analysis system can execute object detection and/or image segmentation. Such analysis may be performed on images of isothermal dendritic growth experiments similar to what is shown in FIG. 4A. Computer vision in the advanced materials analysis combines object detection methods with time series analysis to automatically map the evolution of segments across multiple images and compute growth rates and other time-dependent properties. That way, parameters such as growth velocity and tip radii from dendritic growth can be extracted to determine various properties of the material. These parameters may be different depending on the gravitational conditions.

FIG. 4B shows an original image of a material sample obtained from a video dataset (4B-1), a segmented image from the original image (4B-2), and graphs showing quantitative time-series analysis of the material sample based on the segmented image (4B-3). In FIG. 4B-1, an image of the material sample may be obtained from the video dataset. Image information such as camera angle, resolution, and illumination may be mapped to examine system parameters during data ingestion. Subsequently, image segmentation may be performed as shown in FIG. 4B-2. In FIG. 4B-2, purposeful selection and separation of “objects of interest” (i.e., material boundaries) are determined based on the image information provided in the image in FIG. 4B-1. This provides for automatic edge detection and computation of center of mass in the segmented image in FIG. 4B-2. Subsequently, edge displacement can be monitored to track changes in the material sample over time. Such tracked changes may include changes in area or changes in center of mass, for example. As shown in FIG. 4B-3, the time-series-analysis may be represented by graphs showing how area changes over time or how center of mass changes over time. This data can be used to monitor system evolution to extract time-dependent features such as boundary changes, movements, periodic evolution, frequency changes, anomalous events, etc. These time-dependent features may be dependent on gravity.

FIG. 4C shows a series of images for detecting coarsening in a solid-liquid mixture, including an original image of a material sample indicating particles by white dots (4C-1), a segmented image of the material sample indicating categorizing properties (e.g., size) of the particles based on color, intensity, and shape (4C-2), and an analyzed image after cluster analysis is performed to combine sets of particles based on similar or dissimilar features (4C-3). FIG. 4D shows a graph representative of the cluster analysis performed in FIG. 4C-3. Detection of spherical structures may play an important role in detecting coarsening in solid-liquid mixtures. Computer vision and machine learning techniques may be applied to detect spherical structures (e.g., particles) in an original static image. Detection of the spherical structures and size-based clustering can be performed in FIG. 4C-1. This is shown by white dots in the image in FIG. 4C-1. Thus, as shown in FIG. 4C-1, objects may be classified in a static image based on their physical properties. Subsequently, in FIG. 4C-2, different classes may be highlighted based on their physical properties, such as object size. As shown in FIG. 4C-2, a segmented image of the material sample may be clustered based on size according to three types: small, medium, and large. This classification can be made based on color, intensity, and shape. Subsequently, cluster analysis is performed in FIG. 4C-3 by combining sets/subsets of spherical structures based on similar or dissimilar features. In doing so, one can quantify dynamic processes, structural changes, and properties of objects that evolve over time together with their potential correlations. Dynamic processes, structural changes, and properties of objects that evolve over time together with their potential correlations may be dependent on gravity. This technique performed by computer vision of the advanced materials analysis system enables rapid processing of hundreds or thousands of images/videos/data sets.

FIG. 4E shows an example of a generic phase transition diagram of a material formulation with adjustable parameters that are dependent on gravity levels. Adjustable parameters may include but are not limited to temperature, pressure, concentration, composition, time-temperature-transition, and solubility. Such a phase diagram can be developed for material samples to measure gravity-temperature dependence of viscosity, which may be a critical parameter to the processing and manufacturability of many materials (glasses, thermoset polymers, liquid crystals, etc.). Similarly, concentration-temperature-solubility phase diagrams of relevance to crystallization can be constructed to establish the gravitational impact on delimiting zones between precipitation, nucleation, and supersaturation. Gravitational impact on binary or ternary colloidal systems' composition-temperature phase diagrams may also be important to establish the changes in the spinodal and coexistence curves. These differences may be critically important to the manufacturing of new materials such as optical fibers, membrane proteins, drug design, and pharmaceuticals, among other materials.

FIG. 4F shows an example of a time-temperature-transition diagram that captures the change in stability of a material formulation phase diagram between a 1-g environment and a 0-g environment. The time-temperature-transition diagram of the material formulation in FIG. 4F represents a particularization of FIG. 4E. FIG. 4F highlights the impact of the change in gravitational force (terrestrial-gravity versus zero-gravity) on a time-temperature-transition diagram of a material formulation. As shown in FIG. 4F, the crystallization zone of the material formulation is significantly reduced when the gravitational force is not present. This indicates that in the absence of gravity, the processing regime (area between glass and crystal) is more generous in zero-gravity environments. Also, a glass transition onset occurs at lower temperatures in the zero-gravity environment. A crystal phase of the material formulation is stable in a larger time-temperature regime in terrestrial-gravity environments than zero-gravity environments. A crystallization onset occurs at higher temperatures in the zero-gravity environment.

Experimental setups and procedures for collecting the experimental data may be standardized to ensure consistent data collection. The experimental setups and procedures are defined according to certain processing parameters. In some cases, experiments may be re-run with the same or different material samples to ensure statistical confidence in data collection. As a result, any variations in experimental data can be minimized or at least attributed to hardware issues, user issues, etc.

Database records in the database system may contain a dataset of material properties, such as viscosity, density, phase diagram, etc., associated with a particular material formulation. These datasets may be constructed from the experimental data collected from the non-contact suspension system and datasets of material properties from external data sources. In some embodiments, these datasets may be used in constructing structural or physical models of the material formulation. Such structural or physical models may be added to the advanced materials development system. Or, pre-existing structural or physical models may be built into the advanced materials development system to support the analysis of experimental data collected from the non-contact suspension system. In some cases, quantum mechanical models may be used to predict structure and structural properties of certain material formulations in zero-gravity environments and terrestrial-gravity environments.

In some implementations, the advanced materials development system consolidates experimental data across different experiments for the plurality of material samples, and utilizes algorithms, computer vision techniques, and/or computational models to transform the experimental data into relevant features. The relevant features may include the one or more thermal, mechanical, optical, or structural properties associated with the plurality of material samples, where the aforementioned properties are formatted in a manner that can be stored, organized, and analyzed by the advanced materials development system.

At block 330 of the process 300, an impact of the gravitational force is determined on one or more of a material formulation stability, structure, properties, performance, or manufacturing processing. This impact of the gravitational force on a material and its manufacturing processing may be referred to as “delta-to-gravity.” The impact of the gravitational force may be calculated by comparing macroscopic material properties of a material in a modified-gravity environment against macroscopic material properties of the material in a reference-gravity environment. The reference-gravity environment may be any environment where the gravitational force is different than the gravitational force of the modified-gravity environment. In some embodiments, the gravitational force of the reference-gravity environment is 1-g.

Macroscopic material properties include the one or more thermal, mechanical, optical, or structural properties associated with each of the plurality of material samples. Using machine learning algorithms, computer vision techniques, and/or computational models in the advanced materials development system, the impact of the gravitational force may be calculated. By analyzing the macroscopic material properties in a modified-gravity environment and comparing those properties in a reference-gravity environment, gravitational dependence may be extracted in phase diagrams, TTT diagrams, and temperature-dependent viscosity diagrams. Additionally or alternatively, gravitational dependence may be extracted in material properties such as viscosity, density, surface tension, heat capacity, emissivity, mechanical strength, thermomechanical stresses, etc. Values in the modified-gravity environment (e.g., 0-g) may be compared against values in the reference-gravity environment (e.g., 1-g) to extract the delta-to-gravity. These values may be retrieved from the database system of the advanced materials development system. Thus, in some implementations, the process 300 may further include comparing values associated with the one or more thermal, mechanical, optical, or structural properties of a material formulation in a modified-gravity environment against values associated with one or more thermal, mechanical, optical, or structural properties of the material formulation in a reference-gravity environment.

The advanced materials development system may leverage the impact of gravitational force on thermal, mechanical, optical, and structural properties of a material formulation to determine the impact of gravitational force on any one of the following: stability of the material formulation, properties of the material formulation, structure of the material formulation, performance of the material formulation, and manufacturing processing of the material formulation. Analyzing data of material properties (e.g., thermal, mechanical, optical, and structural properties) with or without gravity can establish a correlation with a material's stability, properties, structure, performance, and manufacturing processing. For example, the impact of gravitational force on thermal, mechanical, optical, and structural properties of a material formulation may be leveraged to determine the impact of gravitational force on the structure of the material formulation. Viscosity, density, phase transitions, or other structure-related parameters may be modeled as a function of gravitational force. Changes in gravity, for example, can affect crystallization dynamics of a material. The advanced materials development system can model such physical processes by comparing structural properties of a material formulation in a modified-gravity environment against a reference-gravity environment.

In some cases, the advanced materials development system may utilize physics/chemistry models such as quantum mechanical models to assist in predicting a behavior of a material formulation in reduced-gravity, hyper-gravity, or zero-gravity conditions. The predictions of the physics/chemistry models may be validated by the experimental data received from the non-contact suspension system. In such cases, the experimental data received from the non-contact suspension system may be used to train physics/chemistry models in the advanced materials development system. The physics/chemistry models may be trained using a model such as a trained neural network, a classification tree, a random forest model, etc.

In some implementations, the impact of gravitational force on thermal, mechanical, optical, and structural properties of a material formulation may be leveraged to determine the impact of gravitational force on the stability of the material formulation. As discussed above, the advanced materials development system can analyze the experimental data and determine a gravitational dependence on a material formulation's properties. In particular, an effect of gravity can be mapped and statistically analyzed for a material formulation's thermal, mechanical, optical, or structural properties, which can be done for several different material formulations. Physics/chemistry models may assist in predicting a behavior of certain material formulations in reduced-gravity, hyper-gravity, or zero-gravity conditions. Using a combination of statistical analysis, machine learning algorithms, and physics/chemistry models, the advanced materials development system may predict a stability of material formulations in reduced-gravity, hyper-gravity, or zero-gravity environments. Machine learning algorithms may iterate over several material formulations across various gravity environments to predict stability based on, for example, a trained neural network model, a Bayes model, or other machine learning model.

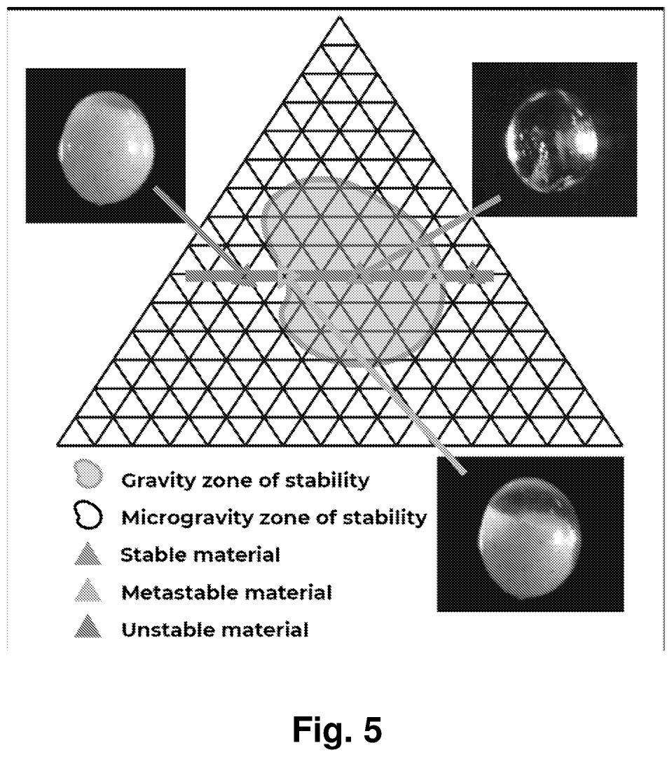

FIG. 5 shows an example of a composition matrix diagram covering a suite of possible material formulations in any percentage combination of three components A, B, and C (AₓB_{y}C_{z}). These components A, B, and C may be relevant to fluorozirconates, fluoroaluminates, fluoroindates, II-VI semiconductor alloys (ZnₓCd_{y}Se_{z}, ZnₓCd_{y}Te_{z}, ZnₓMg_{y}Sc_{z}), NCA (lithium-nickel-cobalt-aluminum oxide), LiCoO₂, LiMn₂O₄, LiFePO₄, NMC, LTO, graphite, LiPF₆, and other materials such as materials relevant to electrolyte solutions, gel polymers, etc. Properties of the material formulation AₓB_{y}C_{z} may be optimized by adjusting composition or by increasing/decreasing a concentration of certain components depending on the desired macroscopic property to be optimized. For example, increasing the percentage of component A may increase mechanical performance, while increasing the percentage of component B may increase optical performance. However, under a terrestrial gravitational environment, these material formulations may become unstable outside of a range of concentrations corresponding to the circular area in the center of the composition matrix diagram. The provided images show that by using terrestrial levitation, a partially stable material formulation when processed in a reduced-gravity environment becomes stable. Stable material formulations AₓB_{y}C_{z} may be obtained by adjusting composition or by increasing/decreasing a concentration of certain components. Under terrestrial-gravity environments, the material formulation AₓB_{y}C_{z} is stable for compositions within the circled area. However, under zero-gravity environments, the material formulation AₓB_{y}C_{z} is stable for compositions beyond the circled area. Using a combination of statistically significant series of experimental data and modeling data, a larger area of stability for the material formulation AₓB_{y}C_{z} is found at zero-gravity environments. This method, however, is not limited to binary, tertiary, or ternary compounds. In theory, this method can be applied to any number of compounds and their corresponding percentage compositions.

In some implementations, the impact of gravitational force on thermal, mechanical, optical, and structural properties of a material formulation may be leveraged to determine the impact of gravitational force on the performance of the material formulation. The performance of materials may be related to their thermal, mechanical, optical, and structural properties. The behavior of the aforementioned properties in reduced-gravity, hyper-gravity, or zero-gravity environments can be modeled using one or more of statistical analyses of processed experimental data, machine learning algorithms, or physics/chemistry models. The advanced materials development system can leverage this modeled data to predict the performance of certain material formulations in a modified-gravity environment or when manufactured in the modified-gravity environment. For example, fiber optic materials may be optimized in performance in terms of its attenuation, flexibility, chromatic dispersion, and mechanical strength. How gravity affects crystallization dynamics in viscous glass and how gravity affects a fiber optic material's glass transition and phase diagram can be predicted and modeled. In some cases, physics/chemistry models may model transport phenomena in porous media in modified-gravity and/or terrestrial-gravity environments. In some cases, physics/chemistry models may model viscosity and fiber pulling in modified-gravity and/or terrestrial-gravity environments. By understanding the effects of microgravity and the effects of removing impurities, a performance-related property such as attenuation in a fiber optic material can be predicted in microgravity.

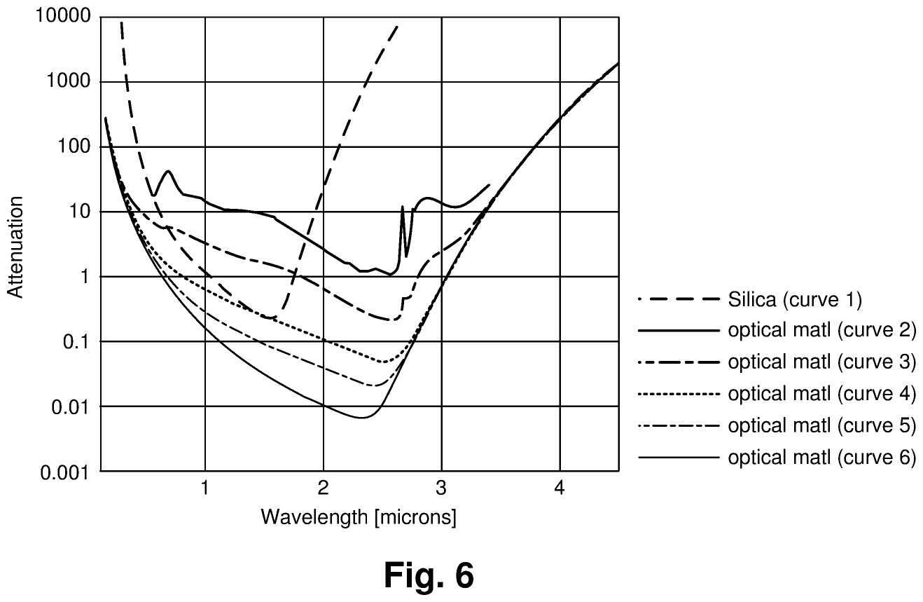

FIG. 6 shows a graph illustrating attenuation at varying wavelengths for fiber optic materials manufactured in terrestrial-gravity environments or zero-gravity environments. As light travels through a medium such as an optical fiber, its intensity is reduced through effects such as absorption or scattering of photons. Attenuation (dB/km) is the ratio of light intensity at a distance (d) relative to light intensity at its source. Attenuation changes for different wavelengths. Reduced attenuation is generally desirable for improved fiber optic material performance. Curve 1 represents optical fiber attenuation performance of silica (glass) manufactured in terrestrial-gravity conditions. Curve 2 represents optical fiber attenuation performance of a commercially manufactured optical fiber in terrestrial-gravity conditions. Curve 3 represents improved optical fiber attenuation performance of the optical fiber manufactured in terrestrial conditions given additional improvements. Curve 4 represents a target attenuation given partial reprocessing in a zero-gravity environment. Curve 5 represents idealized optical fiber attenuation performance of the optical fiber manufactured in space (i.e., zero-gravity) conditions, and Curve 6 represents a theoretical limit for optical fiber attenuation performance of the optical fiber manufactured in space (i.e., zero-gravity) conditions that can only be achieved through new and unique formulations only stable under zero-gravity and using zero-gravity specific manufacturing processes. Each successive curve shows progressive improvement in achieving lower and lower attenuation. The improvement in optical fiber attenuation performance between terrestrial and space conditions shows that microgravity manufacturing results in significantly better attenuation. Without being limited by any theory, the reduced attenuation can be attributed in part to the removal of impurities and improved crystallization dynamics in zero-gravity environments. Controlling one or more parameters in the manufacturing processes in space may further optimize attenuation performance of fiber optic materials.

In some implementations, the impact of gravitational force on thermal, mechanical, optical, and structural properties of a material formulation may be leveraged to determine the impact of gravitational force on the manufacturing processes of the material formulation. The behavior of materials in modified-gravity environments can be analyzed from the experimental data and/or from physics/chemistry models in the advanced materials development system. Models can be developed and trained by the advanced materials development system for various material formulations in modified-gravity environments under different manufacturing conditions. For instance, taking segmented components frame by frame in a video, times series analysis of material formulations at different temperatures in a zero-gravity environment can be analyzed and modeled. This modeled data can be leveraged to determine how space manufacturing conditions for certain material formulations affect material properties, stability, structure, or performance. Machine learning algorithms may be trained to recognize how changes in experimental settings affect critical material properties. That way, manufacturing processes can be optimized for improved product development.

At block 340a of the process 300, a predicted material formulation is identified that is stable in a modified-gravity environment. The predicted material formulation may be based, at least in part, on the impact of gravitational force on thermal, mechanical, optical, and/or structural properties of the plurality of material samples, where the impact of the gravitational force can be calculated by comparing a difference between values at reduced-gravity, zero-gravity, or hyper-gravity environments and values at reference-gravity environments. The advanced materials development system may leverage the impact of the gravitational force on thermal, mechanical, optical, and/or structural properties with physics/chemistry models and machine learning algorithms to identify which material formulations are stable in the modified-gravity environment and which material formulations are unstable in the modified-gravity environment. New materials may be identified that are stable in the modified-gravity environment (e.g., zero-gravity environment) that are not stable in a terrestrial-gravity environment. These new materials that are stable in places such as outer space may be identified using a terrestrial-based non-contact suspension system and terrestrial-based advanced materials development system. As such, it is possible to develop new products in places such as outer space that are not otherwise stable on Earth. Moreover, it is possible to predict the properties, stability, and optimized space manufacturing process on Earth. For example, one or more compositions of heavy metal fluoride glasses may be identified as stable in the zero-gravity environment that is not stable in the terrestrial-gravity environment.

Alternatively, at block 340b of the process 300, a manufacturing process is optimized or a new process is defined for fabricating a material formulation with desired properties in a modified-gravity environment. The desired properties may be related to the thermal, mechanical, optical, or structural properties of the material formulation. Desired properties may be, for example, desired values for material properties such as attenuation, mechanical strength, refractive index, electrical conductivity, etc. As used herein, it will be understood that “desired” properties and “optimized” manufacturing process may be dependent on the preferences, desires, or expectations of the user. The advanced materials development system may specify conditions for manufacturing in the modified-gravity environment. Leveraging the impact of the gravitational force on thermal, mechanical, optical, and/or structural properties with physics/chemistry models and machine learning algorithms, how a material behaves in a modified-gravity environment can be analyzed by the advanced materials development system. This can include how gravitational force impacts the overall phase diagram, scaling laws around glass transitions, and other effects/properties associated with the material. Manufacturing processing parameters and conditions may be specified by the advanced materials development system for achieving desired properties of the material formulation. For instance, sets of experiments and relevant processing conditions (e.g., time, temperature, etc.) are specified to optimize a set of properties for a particular material formulation.

Ordinarily, the presence of gravity restricts the processing conditions in which a material formulation is manufactured. By removing the influence of gravity, it is possible to alter or reconfigure manufacturing processes in fabricating many material formulations. In other words, the manufacturing processing window may be entirely different for material formulations in outer space relative to a terrestrial environment. By way of an example, manufacturing heavy metal fluoride glass fibers can involve: (i) processing fluoride powers to create a limited size preform, and (ii) inserting the preform in a draw tower, and heating the preform to a temperature defined by a glass transition temperature and crystallization temperature to pull a heavy metal fluoride fiber. The aforementioned steps are influenced by gravity. Without the presence of gravity, however, temperature ranges for a heavy metal fluoride glass change, impurity concentrations in a heavy metal fluoride glass change, crystallization dynamics change, devitrification changes, and dynamics of pulling a fiber change, among other changes. Accounting for such changes, the advanced materials development system specifies manufacturing processing parameters for steps of creating a preform and pulling a heavy metal fluoride glass fiber. These steps may be altered or even eliminated in manufacturing in the zero-gravity environment relative to the terrestrial-gravity environment. The manufacturing processing conditions may be optimized for achieving a certain fiber optic performance in the heavy metal fluoride glass. In some cases, manufacturing a fiber optic cable with a desired attenuation may require processing at a certain temperature (T), maintaining a certain viscosity (η), processing for a certain duration of time (t), heating and cooling at a certain rate (r), and maintaining other environmental conditions. These parameters for fabricating the fiber optic cable of a particular composition in a zero-gravity environment may be optimized using the advanced materials development system.

By understanding the gravity-free physics and chemistry that drive processing parameters and material performance, the advanced materials development system can design new materials without gravitational force under the appropriate manufacturing conditions. This can be done terrestrially while avoiding expensive in-orbit trial-and-error experimentation. Certain material formulations of heavy metal fluoride glasses, such as ZBLAN and fluoroindates, may be stable in zero-gravity environments that are not stable in terrestrial-gravity environments. And these material formulations of heavy metal fluoride glasses may achieve a larger band transmission window and reduced attenuation when manufactured under optimized manufacturing conditions in the zero-gravity environment. The advanced materials development system may identify specific material formulations and manufacturing processes for one or more heavy metal fluoride glass formulations in zero-gravity environments with superior mechanical and transmission properties relative to terrestrial-gravity environments.

Various users may access the advanced materials development system that enables them to take advantage of the consolidated experimental datasets, physics/chemistry models, machine learning algorithms, computer vision-based data science, and analytics of material formulations and manufacturing processes in reduced-gravity environments. In some implementations, users may use the advanced materials development system to identify new material formulations that are stable in reduced-gravity, hyper-gravity, or zero-gravity environments and/or to specify manufacturing processing parameters for optimizing properties of material formulations in reduced-gravity, hyper-gravity, or zero-gravity environments. In some implementations, users may use the advanced materials development system to optimize product performance by identifying one or more causes of reduced performance. In some implementations, users may use the advanced materials development system to verify a manufacturing process to identify causes that lead to poor manufactured product performance. In some implementations, users may use the advanced materials development system to verify and validate one or more manufacturing processes by identifying one or more causes of ineffective microgravity manufacturing processes. In some implementations, users may use the advanced materials development system to implement a standardization process that leads to a well-established manufacturing process. In some implementations, users may use the advanced materials development system to support data collection, processing, and analysis so that meaningful insights or analytics can be generated to help identify differences between modified-gravity and terrestrial-gravity investigations/experiments. In some implementations, users may use the advanced materials development system to provide data formatting standardization across microgravity investigations/experiments in a central database system. In some implementations, users may use the advanced materials development system to perform insightful analysis on selected datasets that can lead to potential scientific breakthroughs. In some implementations, users may use the advanced materials development system to analyze sequential images or video frames for extraction of structured data on material formulations in reduced-gravity environments. Such analysis may of images or video frames may be executed using computer vision-based data science techniques as described above.

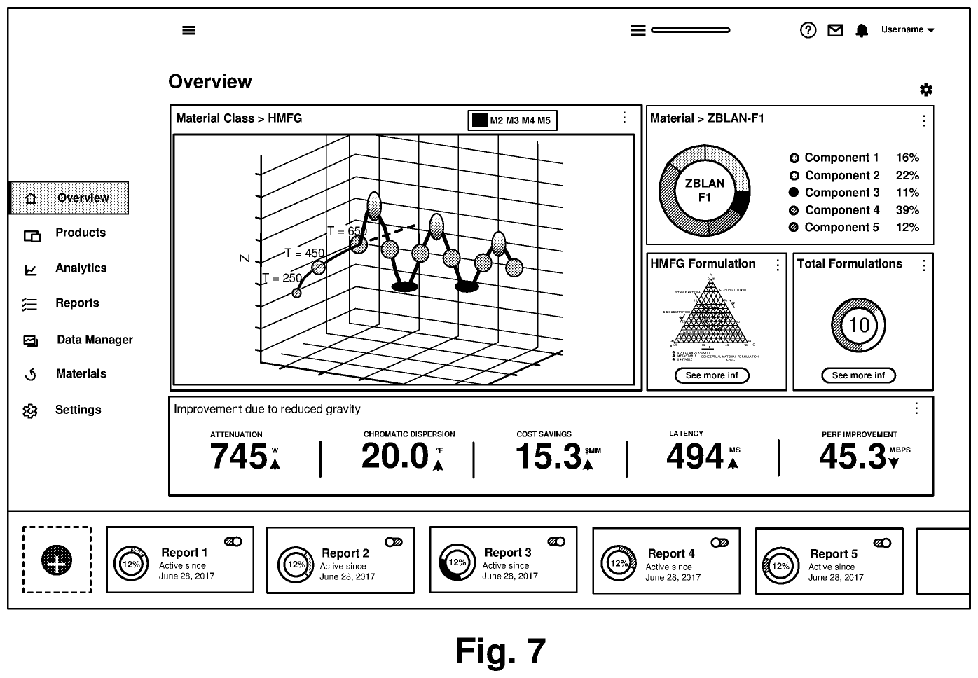

FIG. 7 shows an image of an example user interface displaying reports on a material formulation and data regarding an impact of gravitational force on the material formulation. The user interface may be presented by the advanced materials development system based on interactions with the advanced materials development system by a user. Users may upload experimental data (e.g., experimental images/videos) for a large collection of material formulations in various gravitational environments into a database system of the advanced materials development system. The database system may consolidate the experimental data with data from external data sources. The consolidated data may be collected into database records in the database system, where each database record may include metadata associated with a material formulation, experimental/raw data (e.g., videos/images) associated with the material formulation, properties associated with the material formulation, and modeled data associated with the material formulation. Imaging analysis, time series analysis, simulations performed by physics/chemistry models, and other operations may be performed on the consolidated data to extract useful information regarding the material formulation. Material properties such as viscosity, density, surface tension, heat capacity, emissivity, and mechanical strength may be extracted. Analysis can generate, for example, phase diagrams, TTT diagrams, and temperature-dependent viscosity diagrams. Further analysis may be performed on the consolidated data using the advanced materials development system to extract insights regarding differences in properties/processes for material formulations between terrestrial-gravity environments and reduced-gravity environments. For example, such analysis may generate differences material properties, differences in stability, differences in performance, differences in structure, and differences in manufacturing processes between terrestrial-gravity environments and modified-gravity environments. Some of these differences may be presented to the user in the form of analytics in the user interface.

Users may search the database system to extract insights into how materials behave in modified-gravity environments and how gravity affects a material's performance. Users may initiate a query on specific material formulations. Or, users may initiate a query using search parameters based on material properties, performance criteria, processing conditions, gravitational environments, experimental results, simulation results, and more. Results may be generated and presented to the user that meet the search criteria. When the user accesses a particular database record, information regarding the database record may be presented in the user interface. As shown in FIG. 7, such information may include the composition of a ZBLAN material formulation, phase diagram of the ZBLAN material formulation indicating regimes of stability between terrestrial-gravity and modified-gravity environments, size/shape features as a function of temperature, attenuation improvements in modified-gravity environments, chromatic dispersion improvements in modified-gravity environments, cost savings improvements in modified-gravity environments, latency improvements in modified-gravity environments, and performance improvements in modified-gravity environments. Reports, images, and/or videos may be accessed by the user from the user interface. The reports may include, for example, phase diagrams, TTT diagrams, and temperature-dependent viscosity diagrams. The advanced materials development system may receive a material formulation (e.g., AₓB_{y}C_{z}) as input and provide analytics and other information regarding the properties and behavior of the material formulation in modified-gravity environments.

### Conclusion

A big gap exists between current microgravity research and development efforts and the emerging field of in-space manufacturing. Addressing this gap will require improvements in microgravity product development since products manufactured in microgravity may need to meet both performance and economic requirements. The advanced materials development system provides a versatile and modular tool that allows easy translation of microgravity research and development into marketable products. The advanced materials development system is a platform that offers users a toolset that enables, via customized computer vision (CV) and machine learning (ML) algorithms, a systematic way to design, test, and analyze microgravity experiments. These algorithms are suitable to process a large numbers of sequential images or video frames and perform multidimensional analysis (identification, feature extraction and segmentation, tracking, dimensionality reduction, clustering, classification, time series analysis, state description and evolution, outlier detection, etc.) in a single integrated platform. For example, the platform enables certain users to deploy a series of tools and analyses to expand the interpretation of their own data sets, bringing investigations to clearer conclusions and increasing both science and translational readiness and allowing systematic tracking over several investigations to maximize on cyclical learning. The present disclosure highlights computer vision-based analysis machine learning analysis to a suite of microgravity investigations of myocardial fibers, drosophila behavior, thermocapillary flow, surface tension, melt phenomena, porous media structure, isothermal dendritic growth, coarsening in solid-liquid mixtures, optical fibers, etc. In addition, the present disclosure highlights how the foregoing tools would specifically target the development of physics models to capture the “delta-to-gravity” and enable the prediction of new properties, new materials, and corresponding performance enhancement that aligns with the needs of industrial applications. Though other material designers may apply data science, machine learning algorithms, and/or computer vision techniques, material designers do not leverage such techniques in extracting “delta-to-gravity” and its impact on new material formulations, properties, and predicted performance.

Various user archetypes may access the platform provided by the advanced materials development system. In some embodiments, microgravity principal investigators (PIs) may use the platform's software analytics for microgravity-related experiments. The microgravity PIs may utilize the all-in-one platform for data integration and analytics, may use the platform's advanced data science and computational modeling fine-tuned for microgravity, may use the platform's physics systems simulations, may use the platform's computer vision-assisted design of experiments, and may use the platform's predictive machine learning capabilities. In some embodiments, advanced material designers may use the platform's new materials discovery capabilities. The advanced material designers may utilize the non-contact suspension system to perform experiments terrestrially under microgravity conditions, may utilize the platform to explore stability of new gravity-free material formulations, may utilize the platform to extract “delta-to-gravity” for relevant material properties, may use the platform to optimize performance with machine learning analytics, and may use the platform for market-intelligence-driven prioritization. In some embodiments, space providers may use the platform's software analytics for a seamless experience in microgravity. The space providers may utilize the platform's user-friendly data analytics toolset, may utilize the platform's pre-flight design of experiments optimization, may use the platform's on-the-fly adaptive experimental designs, may use the in-space experiment monitoring, may leverage the platform's inspection, analysis, and certification of in-space manufactured products, and may use the platform's development of new native in-space manufacturing processes.

Although the foregoing disclosed systems, methods, apparatuses, processes, and compositions have been described in detail within the context of specific implementations for the purpose of promoting clarity and understanding, it will be apparent to one of ordinary skill in the art that there are many alternative ways of implementing foregoing implementations which are within the spirit and scope of this disclosure. Accordingly, the implementations described herein are to be viewed as illustrative of the disclosed inventive concepts rather than restrictively, and are not to be used as an impermissible basis for unduly limiting the scope of any claims eventually directed to the subject matter of this disclosure.

What is claimed is:

1. A method of identifying a predicted material formulation that is stable in a reduced-gravity environment, the method comprising:
receiving, at a computing device, experimental data regarding a plurality of material samples from a non-contact suspension system or from an external data source;
processing the experimental data, using the computing device, to determine one or more thermal, mechanical, optical, or structural properties associated with each of the plurality of material samples in a modified-gravity environment;
determining an impact of gravitational force on one or more of a material formulation stability, structure, properties, performance, or manufacturing processing; and
identifying a predicted material formulation that is stable in a modified-gravity environment.

2. The method of claim 1, wherein the one or more thermal, mechanical, optical, or structural properties associated with each of the plurality of material samples in the reduced-gravity environment comprise time-temperature-transition (TTT) diagrams, phase diagrams, and temperature dependent viscosity diagrams.

3. The method of claim 1, wherein the one or more thermal, mechanical, optical, or structural properties associated with each of the plurality of material samples comprise viscosity, density, surface tension, heat capacity, emissivity, mechanical strength, and thermomechanical stress.

4. The method of claim 1, wherein the experimental data regarding the plurality of material samples comprise videos and/or images received from a camera on the non-contact suspension system.

5. The method of claim 4, wherein processing the experimental data comprises segmenting image data into components, mapping segmented components from frame to frame, and extracting feature measurements from the image data to characterize changes in structure and properties of a material sample over time.

6. The method of claim 1, further comprising:
comparing values associated with one or more thermal, mechanical, optical, or structural properties of the material formulation in a reduced-gravity, hyper-gravity, or zero-gravity environment against values associated with one or more thermal, mechanical, optical, or structural properties of the material formulation in a reference-gravity environment.

7. The method of claim 1, wherein the predicted material formulation achieves one or more desired material properties.

8. The method of claim 1, wherein the plurality of material samples include a plurality of heavy metal fluoride glasses (HMFGs) each of different compositions.

9. The method of claim 1, wherein the non-contact suspension system comprises:
primary electrodes in a chamber, wherein an electric field is generated between the primary electrodes to levitate a material sample;
secondary electrodes for stabilizing the material sample in the electric field;
a charge source to charge the material sample so that the charged material sample is levitated in the electric field;
a plurality of lasers for heating the material sample; and
a camera for capturing image data of the material sample.

10. The method of claim 9, wherein the primary electrodes comprise an upper electrode and lower electrode in which the material sample is levitated between the upper and lower electrodes, wherein the secondary electrodes comprise two or more electrodes located in a horizontal plane between the upper and lower electrodes, wherein the charge source comprises a deuterium lamp or ultraviolet (UV) lamp, wherein the plurality of lasers are configured to heat different regions of the material sample, and wherein the material sample has a diameter greater than about 3 mm.

11. A method of identifying a predicted material formulation that is stable in a reduced-gravity environment, the method comprising:
receiving, at a computing device, experimental data regarding a plurality of material samples from a non-contact suspension system or from an external data source;
processing the experimental data, using the computing device, to determine one or more thermal, mechanical, optical, or structural properties associated with each of the plurality of material samples in a modified-gravity environment;
determining an impact of gravitational force on one or more of a material formulation stability, structure, properties, performance, or manufacturing processing; and
optimizing a manufacturing process or defining a new process for fabricating a material formulation with desired properties in a modified-gravity environment.

12. The method of claim 11, wherein the one or more thermal, mechanical, optical, or structural properties associated with each of the plurality of material samples comprise viscosity, density, surface tension, heat capacity, emissivity, mechanical strength, and thermomechanical stress.

13. The method of claim 11, wherein the experimental data regarding the plurality of material samples comprise videos and/or images received from a camera on the non-contact suspension system.

14. The method of claim 11, further comprising:
comparing values associated with one or more thermal, mechanical, optical, or structural properties of the material formulation in a reduced-gravity, hyper-gravity, or zero-gravity environment against values associated with one or more thermal, mechanical, optical, or structural properties of the material formulation in a reference-gravity environment.

15. A non-contact suspension system comprising:
a lower electrode in a chamber;
an upper electrode opposite the lower electrode in the chamber, wherein an electric field is configured to be generated between the upper and lower electrodes;
two or more auxiliary electrodes located in a horizontal plane between the upper and lower electrodes, wherein the two or more auxiliary electrodes are configured to control a position of a material sample in the electric field;
a charge source configured to charge the material sample so that the material sample is levitated in the electric field between the upper and lower electrodes;
a plurality of lasers for heating the material sample; and
a camera for capturing image data of the material sample.

16. The non-contact suspension system of claim 15, further comprising:
a first housing enclosing a plurality of material samples each of different compositions; and
a second housing holding the plurality of lasers, wherein the plurality of lasers include lasers configured to emit different wavelengths.

17. The non-contact suspension system of claim 15, wherein the material sample has a diameter greater than about 3 mm.

18. The non-contact suspension system of claim 15, wherein the plurality of lasers are configured to heat the material sample at different regions.

19. The non-contact suspension system of claim 15, further comprising:
a computing device coupled to the camera, wherein the computing device includes instructions configured to perform the following operations:
receive videos and/or images of the material sample;
process the videos and/or images to determine one or more thermal, mechanical, optical, or structural properties associated with the material sample in a modified-gravity environment; and
determine an impact of gravitational force on one or more of a material formulation stability, structure, properties, performance, or manufacturing processing.

20. The non-contact suspension system of claim 19, wherein the computing device includes instructions further configured to perform the following operation:
identify a predicted material formulation that is stable in a modified-gravity environment, optimize a manufacturing process, define a new process for fabricating a material formulation with desired properties in the modified-gravity environment.

21. The non-contact suspension system of claim 19, wherein the one or more thermal, mechanical, or structural properties comprise time-temperature-transition (TTT) diagrams, phase diagrams, and temperature dependent viscosity diagrams.
